(19) United States
(12) Patent Application Publication
(10) Pub. No.: US 2016/0262681 A1
(43) Pub. Date: Sep. 15, 2016

(54) DETECTING DEPRESSION VIA MOBILE DEVICE DATA

(71) Applicants: AT&T Intellectual Property I, L.P., Atlanta, GA (US); AT&T Mobility II LLC, Atlanta, GA (US)

(72) Inventors: DAVID PATTERSON, Los Altos, CA (US); Jeremy Fix, Acworth, GA (US); Sheldon Kent Meredith, Marietta, GA (US)

(21) Appl. No.: 14/657,707

(22) Filed: Mar. 13, 2015

(51) Int. Cl. A61B 5/16; H04W 24/08; G09B 19/00; H04W 4/02

(52) U.S. Cl. CPC A61B 5/165; H04W 4/028; H04W 24/08; G09B 19/00

(57) ABSTRACT

A method, computer-readable medium, and apparatus for detecting a likelihood of depression of a user are disclosed. A method includes a processor for determining a calling pattern and a mobility pattern of a mobile device of the user during a first time period, detecting a likelihood of depression when the calling pattern of the mobile device during the first time period is indicative of a decline in communications as compared to a reference calling pattern and when the mobility pattern of the mobile device during the first time period is indicative of a decline in movement as compared to a reference mobility pattern, and generating a warning message when the likelihood of depression is detected.

The present disclosure relates generally to a method, computer-readable medium and apparatus for detecting indications of depression, and for providing notifications to responsible entities upon such detection.

### BRIEF DESCRIPTION OF THE DRAWINGS

The teachings of the present disclosure can be readily understood by considering the following detailed description in conjunction with the accompanying drawings, in which:

FIG. 1 illustrates an example system related to the present disclosure;

FIG. 2 illustrates a flowchart of an example method for detecting a likelihood of depression, according to the present disclosure; and

FIG. 3 illustrates a high-level block diagram of a computing device suitable for use in performing the functions, methods, operations and algorithms described herein.

To facilitate understanding, identical reference numerals have been used, where possible, to designate identical elements that are common to the figures.

### DETAILED DESCRIPTION

In one example, the present disclosure discloses a method, computer-readable medium, and apparatus for detecting a likelihood of depression by a processor. For example, the processor may determine a calling pattern of a mobile device of a user during a first time period and determine a mobility pattern of the mobile device of the user during the first time period. The processor may then detecting a likelihood of depression when the calling pattern of the mobile device during the first time period is indicative of a decline in communications as compared to a reference calling pattern and when the mobility pattern of the mobile device during the first time period is indicative of a decline in movement as compared to a reference mobility pattern. The processor may then generate a warning message when the likelihood of depression is detected.

The present disclosure broadly discloses method, computer-readable medium and apparatus for detecting a likelihood of depression of a user of a mobile device. In one example, a likelihood of depression is detected based upon one or more factors including a calling pattern associated with the mobile device and a mobility pattern of the mobile device. For instance, the calling pattern and the mobility pattern of the mobile device during a current time period may be compared to a calling pattern and a mobility pattern of the mobile device from a prior time period. When there is a decline in communications and a decline in movement of the mobile device determined from such comparisons, these factors may be indicative that the user of the mobile device may be depressed. In another example, the calling pattern and the mobility pattern of the mobile device during a current time period may be compared to a calling pattern and a mobility pattern of an average user, or an average user of a same demographic group (e.g., based on age, gender, ethic group, and so on) as the user or in a same geographic area (e.g., within a neighborhood, a school district, a town, a city, a state, a region of a country, a country and so on) as the user. In various examples, the decline in communications may comprise a decline in a number of calls, a duration of calls, or a number of contacts communicated with.

In one example, the calling pattern from the current time period and the prior time period may be derived from call detail records (CDRs) generated by a number of network elements deployed in one or more communication networks. CDRs typically include a calling party, a called party, a timestamp, and other information fields, and are therefore suitable for this purpose. In another example, the calling patterns may be determined by a mobile device from the call logs of the mobile device itself.

The location of the mobile device of the user can be tracked using various device identifiers across various systems. For instance, a location of the user's mobile device may be tracked by device identifier such as an electronic serial number (ESN), a mobile equipment identifier (MEID), and so forth. Using such identifiers that are all associated with the same mobile device, the location and presence of the mobile device can be detected by various network components such as an IEEE 802.11 (“Wi-Fi”) access point, an IEEE 802.16 (“WiMax”) base station, and the like, and cataloged by network components such as home subscriber servers (HSSs), home location registers (HLRs), a network equipment locator service (NELOS), and the like (broadly “cellular location systems”). Alternatively, or in addition, the location of the mobile device may be tracked by the mobile device itself, e.g., using a global positioning system (GPS) integrated with or coupled to the mobile device.

In one example, the likelihood of depression can be quantified by a depression score that may be impacted by a combination of the factors mentioned above. In one example, additional factors may also contribute to a depression score, including: a decline in text message activity as compared to historical text message activity, a decline in social media activity of the user as compared to historical social media activity, a decline in email activity as compared to historical email activity, and so forth. In one example, a warning message may be generated when a likelihood of depression is detected, e.g., when the depression score exceeds a threshold. The warning message may be displayed in a screen of the mobile device, sent to a caregiver of the user, sent to a medical professional, or sent to one or more contacts of the user.

In one example, the mobility patterns used herein may account for a home location of the user and a work location of the user. For instance, the mobility data may indicate that the user is typically travelling between a home location and a work location. Nevertheless, even though the user is deemed to be moving about, such mobility pattern may still be indicative of depression. On the other hand, where mobility data indicates that the mobile device travels to locations that are not related to a work location or a home location, or along the route between these locations, the depression score may be lower.

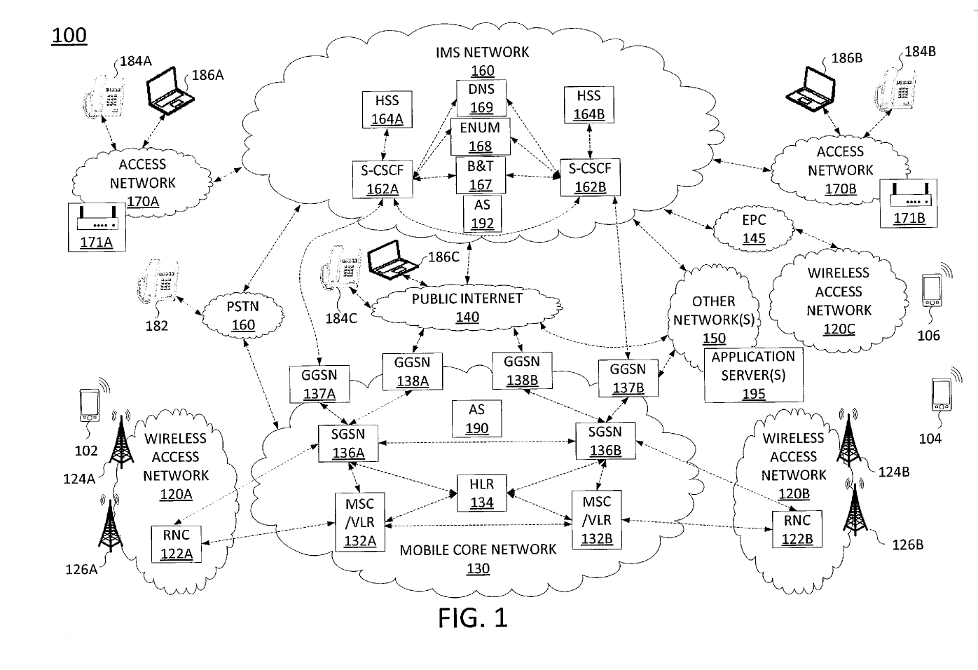

The mobility and calling patterns may also differentiate between weekday and weekend usage. For instance, travels and calls made during the week may be necessary for work. Thus, a lack of travel and a lack of phone calls on weekends are more indicative of depression. In addition, calls to and from close family members may also be ignored or minimized in calculating a depression score insofar as a depressed persons is still likely to have communications with family members, e.g., to coordinate picking up and dropping off children from school, to buy groceries and run other errands, and so forth, while a lack of calls to friends and extended family is more indicative of depression. These and other aspects of the present disclosure are described in greater detail below in connection with the discussion of FIGS. 1-3.

To better understand the present disclosure, FIG. 1 illustrates an example network, or system 100 (e.g., a network having an integrated cellular network architecture), suitable for implementing embodiments of the present disclosure for detecting a likelihood of depression. In particular, system 100 includes example wireless access networks 120A and 120B, and a mobile core network 130 (e.g., a public land mobile network (PLMN)-universal mobile telecommunications system (UMTS)/General Packet Radio Service (GPRS) core network). In one embodiment, each of the wireless access networks 120A and 120B is connected to the mobile core network 130 to provide an integrated cellular network architecture (e.g., a cellular network architecture that includes multi-generational protocols and technologies).

Wireless access networks 120A and 120B may each comprise a radio access network implementing such technologies as: global system for mobile communication (GSM), e.g., a base station subsystem (BSS), GSM enhanced data rates for global evolution (EDGE) radio access network (GERAN), or IS-95, among others, or a UMTS terrestrial radio access network (UTRAN) network, e.g., employing wideband code division multiple access (WCDMA), or a CDMA2000, among others. In other words, wireless access networks 120A and 120B may comprise networks in accordance with any “second generation” (2G) or “third generation” (3G) network technology. Thus, base stations 124A, 126A and 124B, 126B may each comprise a base transceiver station (BTS) or a NodeB. In one example, wireless access networks 120A and 120B may further include radio network controllers (RNCs) 122A and 122B (alternatively referred to as base station controllers (BSCs) in 2G terminology) for managing RF communication of the base stations 124A, 126A and 124B, 126B respectively. RNCs 122A and 122B may perform a variety of wireless network management related tasks such as wireless channel assignments, determining transmission power levels, controlling handovers from one base station to another base station, concentrating multiple signals from mobile endpoint devices for onward transmission to other portions of the respective wireless access networks 120A and 120B, or mobile core network 130, and to perform other functions.

Each of wireless access network 120A and 120B may interface with the mobile core network 130 of system 100 to facilitate communications for various endpoint devices, including mobile devices 102, 104, and 106, each of which may comprise a cellular telephone, a smartphone, a tablet computing device, a laptop computer, a pair of computing glasses, a wireless enabled wristwatch, or any other cellular-capable mobile telephony and computing device (broadly, “mobile endpoint devices”).

In one embodiment, mobile core network 130 includes components of a public land mobile network (PLMN). For example, mobile core network 130 may include one or more mobile switching centers (MSCs) 132A and 132B for each wireless access network that forms part of the system 100. The mobile core network 130 may further include one or more home location registers (HLRs), such as HLR 134, which functions as a central repository of authentication and service validation information, subscription information, and other information pertaining to user subscriptions and services. Similarly, respective visiting location registers (VLRs) may be integrated within each MSC 132A and 132B, which function as temporary repositories of authentication and service validation information, subscription information, and other information pertaining to visiting user subscriptions and services when a user's mobile device is located in a particular geographic region serviced by a particular MSC/VLR. For example, MSC 132A may be designated to serve and administer a first coverage area including wireless access network 120A. Thus, MSC 132A may maintain, e.g., in a visiting location register (VLR), user profile records for mobile devices currently serviced by base stations within the portion of the network that is the responsibility of MSC 132A (e.g., mobile device 102). Similarly, MSC 132B may be designated to administer a second coverage area that includes wireless access network 120B and to perform similar functions to those provided by MSC 132A.

Mobile core network 130 may also include GPRS network elements for handling data calls to and from mobile devices. Such network elements may include serving GPRS support nodes (SGSNs) 136A and 136B, gateway GPRS support nodes (GGSNs) 137A, 138A, 137B, and 138B, and related support components including media servers, application servers, and the like. An SGSN refers to a network node responsible for communicating with mobile devices and routing of data calls. Similar to MSCs 132A and 132B, SGSNs 136A and 136B may have specific coverage areas and be assigned to handle specific wireless access networks of the system 100. A GGSN refers to a network node responsible for the interworking between a GPRS network (e.g., components of core network 130 that support GPRS services and functionality) and external packet switched networks, e.g., the public Internet 140, IMS network 160, and other networks.

In general, in a 3^{rd} generation partnership project (3GPP) network, the setup of a data call may be summarized as follows. A mobile device requests connectivity to an external network or service by providing a corresponding Access Point Name (APN) to the GRPS cellular network architecture. During the Packet Data Protocol (PDP) Context activation phase for a mobile device (e.g., a cellular device, such as any of mobile devices 102, 104, and 106), the SGSN serving the mobile cellular device performs a DNS lookup to determine which GGSN(s) are configured for serving the requested APN for the mobile cellular device. The APN for a large external network may be mapped to a number of GGSNs, while a small external network may only be mapped to a single GGSN. GGSN mapping can also be based on load or mobile cellular device location during this process to improve data service quality. The identities of the mapped GGSN or GGSNs are then returned to the SGSN and the SGSN selects one to create a new PDP Context for the mobile device. In the case of IP network access, at the end of the PDP Context activation process, the mobile device also receives its IP address.

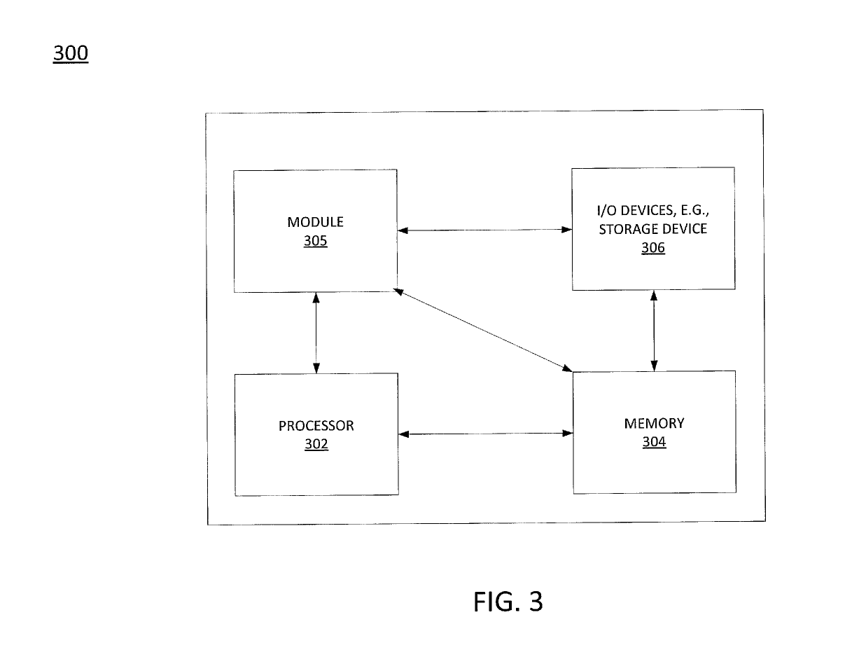

The mobile core network 130 may also include, in one embodiment, an application server (AS) 190. In one embodiment, AS 190 may comprise a computing system, such as computing system 300 depicted in FIG. 3, and may be configured to provide one or more functions for detecting a likelihood of depression, in accordance with the present disclosure. Accordingly, the AS 190 may be connected directly or indirectly to any one or more network elements of mobile core network 130, and of the system 100 in general, that are configured to gather and forward network signaling and traffic data and other information and statistics to AS 190 and to receive instructions from AS 190. In one example, AS 190 is also configured to receive usage information from one or more other application servers (AS) 195 deployed in one or more other networks 150 that may be associated with a social network provider, an email provider, or a similar third-party. AS 190 may also communicate with AS 192 in IMS network 160, as described below. Due to the relatively large number of connections available between AS 190 and other network elements, none of the actual links to the application server are shown in FIG. 1.

In one example, system 100 may further include an IP multimedia subsystem (IMS) network 160. For instance, IMS network 160 may provide VoIP services to users connecting via access networks 170A and 170B which may comprise a Digital Subscriber Line (DSL) network, a broadband cable access network, a Local Area Network (LAN), a wireless LAN (WLAN), a metropolitan area network (MAN), an enterprise network, or other “trusted access network”. In this regard, access networks 170A and 170B may include one or more wireless access points, e.g., 171A and 171B, which may each comprise an IEEE 802.11 (“Wi-Fi”) access point, an IEEE 802.16 (“WiMax”) base station, a Bluetooth access point, a Bluetooth low energy (BLE) beacon or other near-field access point, and the like. The access networks 170A and 170B may be either directly connected to IMS network 160, or indirectly through one or more other networks.

IMS network 160 may also be responsible for routing voice and data calls from cellular networks. For example, calls may enter the IMS network 160 from wireless access network 120C via evolved packet core (EPC) 145, or from wireless access networks 120A and 120B via mobile core network 130. In particular, wireless access network 120C may comprise an evolved UTRAN (eUTRAN) that utilizes IMS network 160 for both voice and data call routing via an all-IP infrastructure.

Notably, mobile core network 130 may be fully equipped to route a voice or data call without invoking IMS network 160. However, in one example, traversal of IMS network 160 may be required to reach a VoIP user via access network 170A or access network 170B, e.g., endpoint devices 184A, 186A, 184B, and 186B respectively, or a 4G cellular user who is on an eUTRAN, e.g., mobile device 106 on wireless access network 120C. For instance, endpoint devices 184A and 184B may comprise Session Initiation Protocol (SIP)-enabled VoIP phones, whereas endpoint devices 186A and 186B may comprise personal computer, laptop computers, and the like, which may be referred to as “softphones.” In another example, additional services may be obtained for cellular users on wireless access networks 120A and 120B via the IMS network, e.g., “VoIP over 3G” services, media services, and so forth.

In one example, access networks 120A and 120B and mobile core network 130 may provide 2G/3G fallback coverage in the event that a 4G/LTE access network is not available, or in the event that a mobile device is not 4G/LTE capable. As such, mobile devices may be registered with both IMS network 160 as well as with cellular core network 130. In this regard, it should be noted that EPC 145 and mobile core network 130 are illustrated as separate networks in FIG. 1. However, in another embodiment mobile core network 130 and evolved packet core (EPC) 145 may comprise a single integrated mobile core network for supporting features of 2-4G access networks, as well as any access network that may utilize still to be deployed or developed technologies. As such, in one example wireless access networks 120A, 120B, and 120C, mobile core network 130, EPC 145 and IMS network 160 (as well as access networks 170A and 170B) may all be controlled by a single network operator.

A first portion of IMS network 160 includes a Home Subscriber Server (HSS) 164A and a Serving—Call Session Control Function (S-CSCF) 162A. A second portion of IMS network 160 includes HSS 164B and S-CSCF 162B. A number of shared resources are also present within IMS network 160: a billing and traffic (B&T) server 167, an ENUM (tElephone NUmbering Mapping) server 168, a domain name service (DNS) server 169 and an Application Server (AS) 192.

An HSS refers to a network element residing in the control plane of the IMS network that acts as a central repository of all customer specific authorizations, service profiles, preferences, etc., with respect to both “home” users and “visiting” users who are temporarily present within a coverage area assigned to a particular HSS. The HSS may also perform user/endpoint device location tracking. Thus, HSS 164A and HSS 164B may store information relating to VoIP users utilizing access networks 170A, 170B as well as cellular users utilizing access networks 120A, 120B and/or 120C. HSS 164A and HSS 164B may also communicate with one another to confirm authorizations and to obtain user/subscriber records to service visiting users. In addition, as mentioned above, mobile devices may be registered with both IMS network 160 as well as with cellular core network 130. Accordingly, HSS 164A and HSS 164B may be configured to receive updates from HLR 134 regarding endpoint device locations and registrations, and vice versa. In accordance, with the present disclosure, current and historic mobility patterns of mobile devices may be tracked using location information from the HSSs and/or HLRs of system 100. For instance, the HSSs and HLRs may provide location information of mobile device to AS 190 and/or AS 192 for storage and compilation into a mobility pattern of the mobile device.

S-CSCFs 162A and 162B reside within the IMS core infrastructure and are connected to various network elements in the IMS network 160 using the Session Initiation Protocol (SIP) over the underlying IMS based core backbone network. S-CSCFs 162A and 162B may be implemented to register users, perform routing and maintain session timers, e.g., for VoIP services. For example, each S-CSCF may also interrogate an HSS to retrieve authorization, service information, user profiles, etc. As illustrated S-CSCF 162A may be configured to utilize HSS 164A, while S-CSCF 162B may be configured to utilize HSS 164B. In addition, S-CSCF 162A and HSS 164A may be delegated a first coverage area while S-CSCF 162B and HSS 164B may be delegated a second coverage area. In one example, these areas may wholly or partially overlap with areas of coverage of mobile core network 130, e.g., the areas assigned to MSC 132A/SGSN 136A and MSC 132B/SGSN 136B, respectively.

In order to complete a call, the S-CSCF may need to interact with ENUM server 168 and DNS server 169 for translating of an E.164 voice network address into an IP address, for example. E.164 refers to an ITU (International Telecommunications Union)-T recommendation which defines the international public telecommunication numbering plan for formatting telephone numbers such that they may be signaled across one or more networks. The E.164 format includes a country code and subsequent digits, but not the international prefix. ENUM refers to a standard protocol defined by the Internet Engineering Task Force (IETF) for translating phone numbers that are in E.164 format to Internet domain names such that a DNS server may resolve the IP addresses for E.164 numbers the same way it resolves traditional website domains. For example, ENUM may be used to transform a phone, a fax or a pager number into a URI (Uniform Resource Identifier).

To illustrate, S-CSCF 163A may translate an original USA phone number of 987-555-1234 to an E.164 formatted number yielding 1-987-555-1234. The E.164 number is then reduced to digits only, e.g., 19875551234. The digits are then reordered back to front, e.g. 43215557891. Once the digits are reordered, dots are placed between each digit and the Internet domain e164.arpa is added to the end. For the above example, the resulting Internet domain is 4.3.2.1.5.5.5.7.8.9.1.e164.arpa.

ENUM server 168 may then be queried by the S-CSCF 162A to resolve on the domain name 4.3.2.1.5.5.5.7.8.9.1.e164.arpa. For each E.164 number that is registered, there may be multiple entries in ENUM server 168 comprising NAPTR (Naming Authority Pointer) resource records, e.g., SIP URIs, which may correspond to a SIP address, a telephone number, a presence service number, an email address, etc.

Each NAPTR resource record contains information pertaining to an order and a preference. In one embodiment, the NAPTR resource records for a particular E.164 number are organized based on the order field and the preference field, e.g., from a lowest order value to a highest order value and from a lowest preference value to a highest preference value. This approach allows a call to be directed to a plurality of possible destinations based upon a preferred order or sequence that can be selectively set by the user.

Furthermore, in one embodiment, each NAPTR resource record may also have an activation field that indicates whether a NAPTR resource record is “active” or “inactive.” An “active” field indicates that the NAPTR resource record can be used, whereas an “inactive” field indicates that the NAPTR resource record should not be used. Thus, this approach allows a user to selectively activate or deactivate a NAPTR resource record.

After retrieving one or more NAPTRs from the ENUM server 168, S-CSCF 162A may then query DNS server 169 for the regular routing of the contact information resided in the NAPTR (Naming Authority Pointer) resource records, e.g., the SIP URI. In sum, after querying HSS 164A and performing the steps of authentication, authorization, and so forth, the S-CSCF 162A will send the ENUM query and the ENUM server 168 will return the NAPTR resource records if the E.164 number is registered, where the S-CSCF 162A then queries the DNS server 169 for the destination of the returned records, e.g., an IP address corresponding to the SIP URI. S-CSCF 162A may then route the call to another S-CSCF or to another network as indicated by the IP address.

The billing and traffic server 167 (broadly a billing server) is a dedicated server that tracks communication traffic for the purpose of generating bills to the customers of the network operator. For example, the billing and traffic server 167 is capable of tracking a large number of call parameters, or features, such as and not limited to: the number of calls, the duration of calls, the calling party numbers, the called party numbers, the types of call, and so on. In the context of the present disclosure a “call,” a “session” or a “communication session” is broadly considered to be any voice or data call (e.g., including short message service (SMS) messages) traversing the network, and includes calls originating and/or terminating in cellular access networks, Wi-Fi networks and other access network. These call features are processed and accounted by the billing and traffic server 167 so that proper bills are generated and provided to the customers of the network operator.

In one embodiment, the network elements that are involved in supporting a call will provide call handling information to the billing and traffic server 167. For example, the network elements (e.g., 122A, 136A, 137A, 162A, 162B) that support a media path between a calling party (e.g., endpoint device 102) and a called party (e.g., endpoint device 186B) will provide one or more call detail records (CDRs) to the billing and traffic server 167 upon termination of the call. CDR records contain multiple fields, including a time stamp, the sender and caller phone numbers, and other network features. Broadly, a CDR is a record produced by a network element containing details of a call that passed through it. CDR records and cause codes conform to an industry standard format. In addition, any and all network elements, including devices/network elements in the access network may generate CDRs in association with a particular call. In the context of the present disclosure, the billing and traffic server 167 may collect CDRs from any and all such network elements, or selected network elements involved in processing one or more calls traversing the networks of system 100.

It should be noted that billing and traffic server 167 is illustrated as a component of IMS network 160. In one example, billing and traffic server 167 may be responsible for collecting CDRs from all network elements that are located both internal and external to IMS network 160. For example, network elements in mobile core network 130, EPC 145, wireless access networks 120A, 120B, 120C, and so forth, may all forward CDRs to billing and traffic server 167. For instance, all of these networks may be managed by the same network operator, or may be managed by two or more different network operators that have agreed to share CDR information. However, in another example, billing and traffic server 167 may alternatively be deployed in mobile core network 130. In still another example, mobile core network 130 and IMS network 160 may both include billing and traffic servers that perform the same or substantially similar functions, or that interact with one another to perform the functions described above.

In one embodiment, AS 192 may comprise a computing system, such as computing system 300 depicted in FIG. 3, and may be configured to provide one or more functions for detecting a likelihood of depression, in accordance with the present disclosure. In one example, AS 192 may perform the same or substantially similar functions to those of AS 190. Thus, the AS 192 may be connected directly or indirectly to any one or more network elements of IMS network 160, mobile core network 130, and of the system 100 in general, that are configured to gather and forward network traffic data (e.g., CDRs or information derived from CDRs) and other information and statistics (e.g., mobile device location information) to AS 192, and to receive instructions from AS 192. In one example, AS 192 may also be configured to receive usage information (e.g., social media usage information, email usage information, and so forth) from one or more other application servers (AS) 195 deployed in one or more other networks 150. In one example, AS 190 and AS 192 may cooperate to perform various aspects of the present disclosure in a distributed manner. Due to the relatively large number of connections available between AS 192 and other network elements, none of the actual links to the AS 192 are shown in FIG. 1.

Each of the mobile core network 130 and IMS network 160 may also interface to a public-switched telephone network (PSTN) 160, to enable communications with PSTN endpoints, e.g., endpoint device 182, and public Internet 140, to enable communications with devices accessible via the Internet, e.g., endpoint devices 184C, 186C, which may comprise a VoIP phone and a softphone, respectively. For example, VoIP users who are not subscribers of IMS network 160 or who are not on a “trusted access network” may nevertheless send and receive calls involving VoIP endpoint devices of access networks 170A and 170B and/or mobile endpoint devices on wireless access networks 120A, 120B, and 120C via the public Internet 140.

In accordance with the present disclosure endpoint device 102 may be associated with a first user. Endpoint device 102 may comprise any type of cellular-capable mobile telephony and computing device (broadly, a “mobile device”). Thus endpoint device 102 is equipped with at least one cellular radio/transceiver and may connect with mobile core network 130 and/or IMS network 160 via any one or more of wireless access networks 120A, 120B and 120C. Endpoint device 102 may also be equipped for any number of different modes of communication. For instance, endpoint device 102 may be further equipped with an IEEE 802.11 transceiver, an IEEE 802.16 transceiver, a Bluetooth transceiver, and so on. In this regard, endpoint device 102 may communicate with mobile core network 130 and/or IMS network 160 via any one or more of these different modes of communication. For instance, endpoint device 102 may travel such that endpoint device 102 connects to IMS network 160 via access network 170A. In one example, access network 170A may comprise an open Wi-Fi network, the user's home Wi-Fi network, or a Wi-Fi network that is provided by the same network operator that manages IMS network 160, mobile core network 130, etc. Similarly, endpoint device 102 may connect with mobile core network 130 and/or IMS network 160 via the Internet 140.

In accordance with the present disclosure, AS 190 and/or AS 192 may obtain CDRs directly from various components of the system 100 or obtain aggregate records from the billing and traffic server 167. Similarly, AS 190 and/or AS 192 may obtain location information from the HSSs and/or HLRs of system 100. In turn AS 190 and/or AS 192 may generate and store current and historic calling patterns and mobility patterns of mobile devices derived from the CDRs and mobile device location information, respectively.

AS 190 and/or AS 192 may track the location of endpoint device 102 in a variety of ways in conjunction with the system 100. For example, a location of endpoint device 102 may be tracked by device identifier such as an electronic serial number (ESN), a mobile equipment identifier (MEID), an integrated circuit card identifier (ICCID), an international mobile equipment identity (IMEI) or an international mobile subscriber identity (IMSI), a telephone number, e.g., an E.164 number or a number that is translatable into E.164 format, a media access control (MAC) address of a Wi-Fi transceiver, WiMax transceiver, Bluetooth transceiver, and the like, or an Internet Protocol address.

Notably, AS 190 and AS 192 may not actually perform device registrations with the respective networks. Thus, for example, a registration or other message containing device identification information and/or device location information may be received by AS 190 and/or AS 192 as a copy from one or more other network elements that are actively involved in the forwarding and the processing of the registration. Alternatively, or in addition, the location of endpoint device 102 and other endpoint devices may be tracked by one or more home subscriber servers (HSSs), home location registers (HLRs) a network equipment locator service (NELOS) and the like (broadly “cellular location systems”), where the location(s) stored by the respective devices are accessed by AS 190 and/or AS 192 in response to a query from AS 195 in card issuer network 150.

To illustrate, one or more of the device identifiers may be contained in a registration message, a session management message, a response to a paging message, a location update message, or similar message from the endpoint device 102, and may be received via one or more network elements such as gateways/border elements, a P-CSCF, an S-CSCF, an MSC, etc. Depending upon the particular access network and/or the particular connection method utilized by endpoint device 102, different device identifiers may be received for endpoint device 102 at different times. For instance, if endpoint device 102 is in a remote area without cellular coverage or if cellular radio(s) are turned off, but there is currently Wi-Fi access, the device identifier may include the Wi-Fi MAC address and/or an IP address. If endpoint device is in another area with only cellular coverage, the device identifier may not include the Wi-Fi MAC address, but may include the ESN, MEID, IMSI, and so forth.

The location may be contained explicitly within the message, e.g., global positioning system (GPS) location information included in the message from endpoint device 102, serving base station information that is appended to a message by the base station or an MSC, and so forth, or may be determined based upon the route taken by the message and the network device(s) from which the message is received. In one example, endpoint device 102 may connect to system 100 via a proxy device, such as a personal base station, mobile hotspot, a femtocell, and the like. Thus the location of the proxy device may be determined or approximated by AS 190, AS 192 or other component of system 100, and the location of endpoint device 102 is assumed to be at or near the same location that is determined for the proxy device.

For example, base stations, wireless access points, and the like may have known locations which are stored by AS 190 and/or AS 192 such that the approximate location of endpoint device 102 may be inferred based upon the point at which the endpoint device 102 connects to the network. In another example, the approximate location may be determined from the current IP address endpoint device 102. For example, the IP address can be cross-referenced to a database that contains geographic approximations for various blocks of assigned IP addresses. In another example, the location may be determined by comparing relative timing offset information of different mobile endpoint devices that are communicating via the same cellular base station(s) as endpoint device 102. Thus, the location of endpoint device 102 can be determined by AS 190 and/or AS 192 in a variety of ways, and may be stored directly by AS 190 and/or AS 192, or may be stored in an HSS, a HLR, a NELOS system, or other storage location within system 100.

In one example, AS 190 and/or AS 192 may broadly track user presence data for supporting the detection of a likelihood of depression with respect to the various users, where the presence data may include location data as described above. For example, AS 192 may tracking a large number of presence parameters associated with a user or a particular user account, such as: internet protocol address(es) of user device(s), current cell tower, base station or wireless access point, instant messaging service status, social networking status, device state(s), such as on, off, standby, sleeping, etc., recent call/messaging usage information, global positioning satellite (GPS) location information, device speed information, availability information (e.g., based upon calendar, instant messaging status, or the like), current device identifier information (e.g., where a user has multiple devices registered in the same user account and/or where the user subscribes to a follow-me service or the like), device access time information, and so on. These call features may thus be processed and stored by AS 192 for use by AS 192 or other components of the system 100 for detecting a likelihood of depression with respect to one or more users.

In one embodiment, the network elements that are involved in supporting calls, messages and registrations of the network elements will provide presence information to AS 192. For example, a user may own endpoint device 102, which may comprise a cellular telephone, and may also own endpoint device 186A, which may comprise a laptop computer. Consider where the S-CSCF 162A may register endpoint device 186A with the IMS network 160. Since the endpoint device 186A (i.e., the laptop computer) is currently registered with the S-CSCF 162A, the S-CSCF 162A therefore knows that the user is currently present on his or her laptop computer 186A and is accessible via access network 170A. Accordingly, this presence information may be passed by the S-CSCF 162A to AS 192. In the same manner, any and all network elements, including devices/network elements in the access networks, may generate presence information of a user and/or device in association with a particular call, message or registration. For example, a base station in a cellular access network (e.g., wireless access network 120A) may indicate that one of the user's other devices, e.g., endpoint device 102, is currently serviced by the base station 124A. As such, presence information indicating that the endpoint device 102 is serviced by this particular base station 124A may be passed to AS 190 and/or AS 192. This particular type of presence information indicates both an approximate geographic location of a user as well as the user's presence on the cellular network via the user's cellular endpoint device (as opposed to the user being present on a home or work computer, a wireless access network, VoIP phone, etc.). In the context of the present disclosure, the AS 190 and/or AS 192 may collect such presence information from any and all such network elements, or selected network elements involved in network 100. In one embodiment, the AS 190 and/or AS 192 polls network devices to obtain user presence information. In another embodiment, various network elements automatically send presence information to the AS 190 and/or AS 192 (e.g., according to a scheduled, at the end of each call/message/session, etc.).

In one example, one or more application server(s) 195, or one or more other components of other network(s) 150 may also track and store current and historical usage information in connection with email or social network accounts. For example, if the user of endpoint device 102 logs in to an email account, one of the other network(s) 150 may create an entry in a database that stores the date and time of the login, an identification of a device from which the login was received, a location from which the login was received, and so forth. In addition, the one of the other network(s) 150 may further track a number of outgoing emails, a number of incoming emails, the number of contacts with whom the user has communicated, and so forth during one or more time periods. Similar information may also be obtained from another one of the other network(s) 150 that may comprise a network of a social media provider. For instance, one of the application server(s) 195 of a social media provider network may track logins to a user account, incoming and outgoing contact messages, posts, links to other media, favorites, likes, and so forth. Notably, in one example, this historical email or social media usage information can be provided by the one or more application servers 195 to AS 190 and/or AS 192, either periodically, or in response to a request from AS 190 and/or AS 192, for use as additional factors in calculating a depression score and detecting a likelihood of depression of a user.

In addition, some users may almost always carry their mobile devices with them at all times, whereas other users may often leave their mobile devices at home or in the car. It is more probable that such users will have a depression score that will cross the threshold, since a lack of mobility of the mobile device may be assumed to be a lack of mobility of the user. Thus, additional presence information may be used to determine that the mobility data of the mobile device is not a reliable indicator of the likelihood of depression of the user. For example, if a user logs in to an email account or social media account from a work computer, from a computer at a hotel, and so forth, this may indicate that the user has travelled and that the mobile device may simply have been left at home.

The foregoing description of an integrated cellular network architecture of system 100 is provided as an illustrative example only. In other words, the example of system 100 is merely illustrative of one network configuration that is suitable for implementing embodiments of the present disclosure. As such, other logical and/or physical arrangements for the system 100 may be implemented in accordance with the present disclosure. For example, network components, such as MSCs 132A and 132B, may be included in respective wireless access network portions (e.g., wireless access networks 120A and 120B) instead of being deployed within the mobile core network 130, or in other portions of system 100 that are not shown, while providing essentially the same functionality. As another example, DNS server 169 may reside in mobile core network 130 or may comprise a public DNS server hosted by another entity on the public Internet 140. Similarly, system 100 may comprise multiple DNS servers, multiple ENUM servers, and so forth. Thus, these and other modifications are all contemplated within the scope of the present disclosure.

In addition, various elements of wireless access networks 120A, 120B, and 120C, EPC 145, mobile core network 130 and IMS network 160 are omitted for clarity, including gateways or border elements providing connectivity between such networks. Similarly, mobile core network 130 and IMS network 160 may each run atop an Internet Protocol/Multi-Protocol Label Switching IP/MPLS network infrastructure, the details of which are also omitted from FIG. 1.

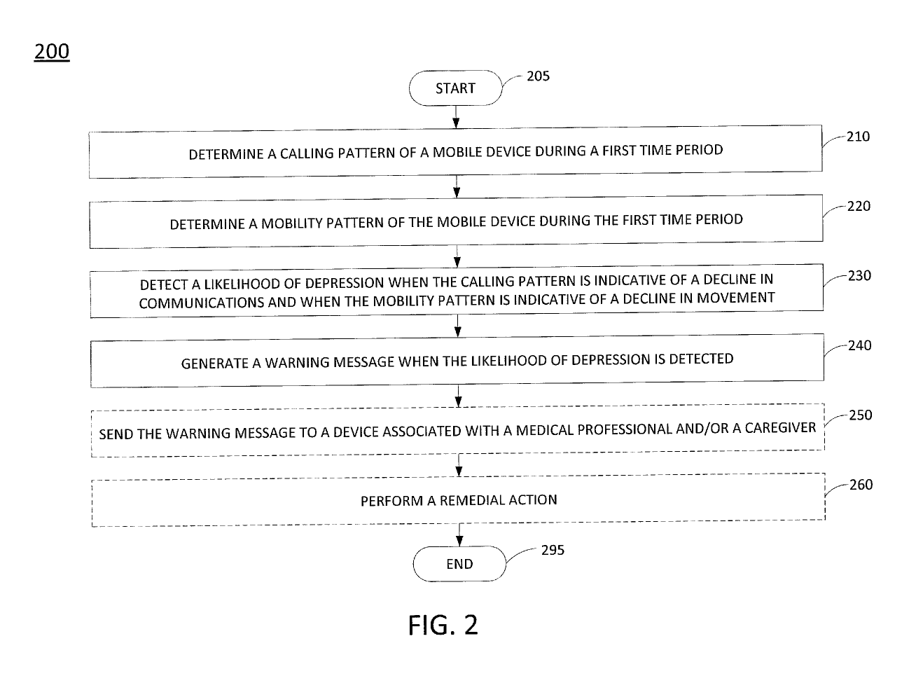

FIG. 2 illustrates a flowchart of a method 200 for detecting a likelihood of depression. In one embodiment, the steps, operations or functions of the method 200 may be performed by any one or more of the components of the system 100 depicted in FIG. 1. For example, in one embodiment, the method 200 is performed by THE application server 190 or 192. In another embodiment, the method 200 is performed by application server 190 together with application server 192 in a distributed manner and in coordination with one another. In still another embodiment, the method 200 may be performed by a mobile device, such as mobile device 102 in FIG. 1. Alternatively, or in addition, one or more steps, operations or functions of the method 200 may be implemented by a computing device having a processor, a memory and input/output devices as illustrated below in FIG. 3, specifically programmed to perform the steps, functions and/or operations of the method. Although a mobile device and any one of the elements in IMS network 160 or mobile core network 130 may be configured to perform various steps, operations or functions of the method 200, the method will now be described in terms of an embodiment where steps of the method are performed by a processor, such as processor 302 in FIG. 3.

The method 200 begins in step 205 and proceeds to step 210. In step 210, the processor determines a calling pattern of a mobile device during a first time period. In one example, the calling pattern may be determined from call detail records (CDRs) generated and stored in connection with voice calls, data calls, text messages, and so forth of the mobile device. In various examples, the calling pattern may comprise, with respect to the first time period, factors such as: an average number of calls, an average call duration, a total duration of calls, a number of unique contacts communicated with, a number of outgoing calls, a number of outgoing calls to contacts who are not close family members or work colleagues, and so forth. The first time period may comprise a day, a week, a two-week period, a month, and so forth. However, it should be understood that the present disclosure is not limited to any particular time period. In one example, the calling pattern may comprise a composite of two or more of the above factors. For instance, the present disclosure may calculate a depression score based upon various factors, as described in greater detail below.

At step 220, the processor determines a mobility pattern of the mobile device during the first time period. In various examples, the mobility pattern may comprise a distance travelled during the first time period, a time spent away from a home area and a work area during the first time period, a distance travelled during the first time period and excluding travels between the home area and the work area, and so forth. In one example, the mobility pattern may comprise a composite of two or more of the above factors. For instance, the present disclosure may calculate a depression score based upon various factors, as described in greater detail below.

In one example, the mobility pattern may be determined based upon location data stored by a communication network. For instance, the processor may obtain location data from one or more application servers, home subscriber servers (HSSs), home location registers (HLRs) a network equipment locator service (NELOS) and the like, which may track the location(s) of a mobile device. In another example, the mobility pattern may be based upon location data of a mobile device that is provided by the mobile device itself. For instance, the processor performing the method 200 may comprise a processor of a mobile device that is equipped with a GPS. Thus, the tracking of locations of the mobile device and the determination of the mobility pattern may be performed without assistance from any network-based devices (i.e., using only components of the mobile device). Alternatively, the processor performing the method 200 may comprise a processor of a network-based device that receives the location data from the mobile device.

Broadly, all sources of information regarding calling patterns and mobility patterns of mobile devices in accordance with the present disclosure may be referred to as “mobile device data.” It should also be noted that where the term “first time period” is used in reference to the calling pattern and the mobility pattern of the mobile device, the time period with respect to the calling pattern may be slightly different from the time period with respect to the mobility pattern. For example, a calling pattern may be based upon data from a full week, whereas the mobility pattern may be based upon data that omits a portion of the full week, e.g., omitting four hours from late evening of Wednesday into early Thursday morning and so on, but otherwise includes the full week. In other words, mobility pattern can be tracked within a subset of the total time frame for the full week, but will still be considered to be the mobility pattern for the full week, e.g., omitting the time period in which most individuals are sleeping. Nevertheless, both time periods may be referred to as the “first time period” as the durations are substantially identical and will not substantially impact the effectiveness of the present method for detecting a likelihood of depression.

At step 230, the processor detects a likelihood of depression when the calling pattern is indicative of a decline in communications and when the mobility pattern is indicative of a decline in movement. In one example, the decline in communications is determined based upon a comparison of the calling pattern in the first time period to a reference calling pattern. In one example, the reference calling pattern is based upon historical call detail records of the mobile device during a second time period. For example, the first time period may comprise a current month or a most recent month, while the second time period may comprise a prior month, a prior year, and so forth. To illustrate, in one example the first time period may comprise a month, while the second time period may comprise a year. In addition, in the present example, the calling pattern in the first time period may comprise a number of calls per week over the course of the month, while the reference calling pattern may comprise an average number of outgoing phone calls per week over the course of the year of the second time period. As such, the first time period and the second time period may comprise the same duration of time, or may comprise different durations of time.

In one example, the average number of calls (for both the first time period and the reference calling pattern/second time period) comprises an average number of outgoing calls. In particular, a user who is suffering from depression may still receive incoming calls but is less likely to proactively call others. In another example, the calling pattern in the first time period and the reference calling pattern each comprises an average number of minutes of calls during a time interval. In one example, the average number of calls relates to calls involving non-family members and/or non-work contacts of the user. Notably, a user may still engage in calls that are necessary to coordinate family responsibilities and work responsibilities, but may be less likely to engage in non-essential communications relating to social activities with friends, calls without a specific purpose, and so on. In still another example, the calling pattern comprises an average number of minutes of calls during a time interval. For example, a user may spend an average of 30 minutes engaged in calls per week during a current month, while historically the user has spent an average of 100 minutes per week engaged in calls over the course of a prior year. As such, this decline in communications may be considered indicative of a likelihood of depression. In one example, the average number of minutes of calls of the first time period and the average number of minutes of calls of the reference calling pattern may relate to only outgoing calls, or may relate only to calls involving contacts who are not close family members or work contacts.

In one example, the reference calling pattern may be wholly or partially based upon historic call detail records (CDRs) of a plurality of mobile devices during a second time period. In other words, the reference calling pattern is not necessarily limited to the calling pattern of the same mobile device of the user, but instead may comprise a calling pattern that relates to an average user. In one example, the plurality of mobile devices may be mobile devices of users in a same demographic group as the user. For instance, if the user is male, the reference calling pattern may be derived from historic CDRs of only devices of male users. Similarly, if the user is between the ages of 45-55, the reference calling pattern may be derived only from CDRs of devices of male users in this age range.

In one example, the decline in movement is determined based upon a comparison of the mobility pattern in the first time period to a reference mobility pattern. In one example, the mobility pattern in the first time period is determined from mobility records of the mobile device during the first time period. Accordingly, in one example, the reference mobility pattern may be determined from historical mobility records of the mobile device during the second time period. In another example, the reference mobility pattern is determined from historical mobility records of a plurality of mobile devices during the second time period. In other words, the reference mobility pattern is not necessarily limited to the mobility pattern of the same mobile device of the user, but instead may comprise a mobility pattern that relates to an average user, or to an average user within a same demographic group and/or a same home geographic area as the user of the mobile device.

In one example, the mobility pattern in the first time period may comprise a total distance travelled over the course of the month, while the reference mobility pattern may comprise an average distance travelled in a one month period over the course of the second time period (e.g., an entire year, a two year period, and so forth). In another example, the mobility pattern in the first time period may comprise a total time spent outside the areas surrounding the user's home and work locations over the course of the month, while the reference mobility pattern may comprise an average time spent outside the areas surrounding the user's home and work locations during a one month period over the course of the second time period.

The reference mobility pattern may take into account that different users may have vastly different commutes to and from work, school, and the like. Thus, the reference mobility pattern may exclude weekdays, or only include weekends and evenings, the reference mobility pattern may only be based upon movements of mobile devices of users who live in the same town or within a certain distance from the user's home (as determined based upon the billing address or registered service address of the user, or as inferred from the most frequent serving base station or GPS coordinates of the user's mobile device during typical sleeping hours), and so on.

It should be noted that in one example, the detecting of the likelihood of depression may comprise calculating a depression score from a number of factors. For instance, the depression score may be based upon a comparison of the calling pattern of the mobile device during the first time period with the reference calling pattern and further based upon a comparison of the mobility pattern of the mobile device during the first time period with the reference mobility pattern. In one example, when the depression score exceeds a threshold, then the likelihood of depression may be detected. In addition, the depression score may be calculated from a plurality of weighted factors, including a first factor for decline in communication and a second factor for decline in movement. In one example, the depression score may be calculated according to the following equation:

DS=X·DC+Y·DM+Z₁·A₁+Z₂·A₂+ . . . +Zₙ·Aₙ EQUATION 1

where DS is the depression score, DC is the decline in communications, DM is the decline in movement, A₁ to Aₙ are a number of possible additional factors, and X, Y, and Z₁ to Zₙ are weightings that may be applied to each of the factors to generate the depression score, DS.

To illustrate, the comparison of the calling pattern of the mobile device during the first time period with the reference calling pattern may indicate that there is a 30 percent decline in communications. Thus, DC=0.30. The comparison of the mobility pattern of the mobile device during the first time period with the reference mobility pattern may indicate that there is a 50 percent decline in movement. Thus, DM=0.50. If X=60, Y=40, and no additional factors are used, then the depression score DS=(60) (0.30)+(40) (0.50)=38. Accordingly, if the threshold for detecting a likelihood of depression is 35, then the processor may determine that the user of the mobile device is likely depressed, since the depression score of 38 exceeds the threshold. It should be noted that this example is simplified for illustrative purposes and that the factor weightings, threshold, and other aspects are not necessarily indicative of depression. It should also be noted that the depression score may comprise a range that is not tied to any particular convention, such as a 0 to 100 scale. Thus, the factor weights need not sum to 1, or 100 percent. The threshold can be generated or established based on a statistical analysis performed on a large number of subscribers who are deemed to be not suffering from depression.

In one example, an additional factor that may be used in calculating a depression score may comprise a comparison of a number of email messages from the first time period with a reference number of email messages (from the second time period). Still another factor may comprise a comparison of a level of social media activity from the first time period with a reference level of social media activity (from the second time period). Another factor may comprise a comparison of a number of text messages from the first time period with a reference number of text messages (from the second time period). However, it should be noted that in one example, text messages and voice calls may both be utilized to generate the “calling pattern” of the first time period and the reference calling pattern. In other words, texts and voice calls may be treated as a single factor.

In addition, as mentioned above, the calling pattern from the first time period and the reference calling pattern may relate only to outgoing calls, only to calls with contacts that are not close relatives or work contacts, and so forth. However, in calculating a depression score, these different examples may all be used as different factors that are used to calculate the same depression score. For example, one factor may comprise a comparison of the calling pattern of the mobile device during the first time period with the reference calling pattern with respect to all calls incoming and outgoing from the mobile device, while a second factor may comprise a comparison of the calling pattern of the mobile device during the first time period with the reference calling pattern with respect to only outgoing calls. Still a third factor may comprise a comparison of the calling pattern of the mobile device during the first time period with the reference calling pattern with respect to both incoming and outgoing calls, but only with respect to non-family members and non-work contacts, and so on.

Various additional factors may be utilized in a similar manner with respect to the mobility pattern in the first time period and reference mobility pattern. For example, one factor may comprise a comparison a total distance moved in the first time period with a total distance moved in a reference mobility pattern (from the second time period), while another factor may comprise a comparison of only distances moved during weekends and evenings in the mobility pattern in the first time period and in the reference mobility pattern.

It should also be noted that the present disclosure is not limited to calculating a depression score according to the above equation, nor is the present disclosure limited to using only the abovementioned factors. For instance, additional factors may further include the number of contacts in a user's calling network, the depression scores of the user's contacts, the number of locations visited by the mobile device/user that correspond to saloons, and so on. In one example, a likelihood of depression may be determined using a decision tree, or similar model or algorithm. To illustrate, a decision tree may utilize various factors, such as those mentioned above, as decision points within the tree structure, with the leaves of the tree resulting in a decision as to a likelihood of depression. In one example, the results may be simply: “likely depressed” or “not likely depressed.” In another example, the results may have several levels of gradation, such as: “very likely depressed,” “likely depressed,” “unlikely depressed,” “highly unlikely depressed,” and so forth. In still another example, the decision tree results may comprise numeric scores. Accordingly, in the context of the present disclosure, a “depression score” may comprise a decision tree result according to one of the foregoing examples. Thus, the present disclosure broadly encompasses the use of a wide array of parameters derived from network call and location data that are subjected to a number of possible mathematical treatments for optimal detection of the onset or worsening of depression.

At step 240, the processor generates a warning message when the likelihood of depression is detected. For example, the warning message may contain a notification that the mobile device data is indicative of depression of the user. In one example, the warning message may include the depression score, or a confidence score that is derived from the depression score. In one example, a relative scale may be provided in the warning message in addition to the depression score. For instance, if the depression score is not on a percentage-based scale, a depression score of 225 may have no meaning unless a minimum, maximum, and/or range indicators (e.g., 0-175, not likely depressed, 175-200, depression possible, and 200-250, depression likely) are provided in addition to the overall depression score.

At optional step 250, the processor sends the warning message to a device associated with a medical professional or a caregiver. For instance, the user may pre-register one or more medical professionals that are authorized to receive protected health information (PHI) associated with the user. For example, the user may provide a phone number or email address associated with a medical professional that may be stored by a mobile device or network-based application server, e.g., depending upon where the processor performing the method 200 is deployed. As mentioned above, the detection of the likelihood of depression may include calculating a depression score and comparing the score to a threshold. Accordingly, in one example, step 250 may include providing the depression score to the medical professional as part of the warning message.

In this regard, it should be noted that ranges of depression scores that are deemed to be indicative of depression may be adjusted based upon feedback from medical professionals and/or users themselves. For instance, a number of users who are found to be likely suffering from depression based upon the present method may ultimately visit a medical professional for treatment and confirmation of the diagnosis. However, a number of the users may be found to not actually be suffering from depression after a medical evaluation. Thus, feedback from medical professionals may be used to adjust the threshold(s) or range(s) utilized to determine a likelihood of depression. For example, if the processor determines that depression scores of 200-250 are indicative of a likelihood of depression, but feedback from medical professionals indicates that this range is too low, the processor may adjust the range such that depression scores of 245-295 are instead the minimum scores that will lead to a determination that depression is likely.

In another example, the warning message may be sent to a caregiver (e.g., a parent, a guardian, a relative of the user, a school official and so on) of the user. For instance, the user may be a child or an elderly family member for whom a relative has provided the mobile device. As such, contact information for the caregiver, e.g., an email and/or telephone number, may be stored as an emergency contact within the mobile device, or within subscriber information stored on a network-based device. For example, a mobile device may have one or more special contact entries for “in case of emergency” (ICE) that can be used by for a number of purposes.

At optional step 260, the processor performs a remedial action in response to the detection of the likelihood of depression. For example, the processor may cause humorous, positive, uplifting, or supportive media to be presented on the mobile device for the user. The processor may also cause contact information for medical professionals, articles or links to articles regarding techniques to fight depression to be presented, and so on. In still another example, the processor may present one more messages on the mobile device suggesting that the user call or write to one or more contacts of the user. Alternatively, or in addition, the processor may send a message to one or more contacts of the user, suggesting that the contacts call or write to the user. The message may or may not include the reason for the suggestion to contact the user. Following any one of steps 240-260, the method 200 proceeds to step 295 where the method ends.

In addition, although not specifically specified, one or more steps, functions or operations of the method 200 may include a storing, displaying and/or outputting step as required for a particular application. In other words, any data, records, fields, and/or intermediate results discussed in the respective methods can be stored, displayed and/or outputted either on the device executing the method 200, or to another device, as required for a particular application.

Furthermore, steps, blocks, functions or operations in FIG. 2 that recite a determining operation or involve a decision do not necessarily require that both branches of the determining operation be practiced. In other words, one of the branches of the determining operation can be deemed as an optional step. In addition, one or more steps, blocks, functions or operations of the above described method 200 may comprise optional steps, or can be combined, separated, and/or performed in a different order from that described above, without departing from the example embodiments of the present disclosure.

As such, the present disclosure provides at least one advancement in the technical field of detecting and preventing depression. In particular, the present disclosure enables automatic detection of patterns that are indicative of depression by tracking the locations and movements of a user's mobile device, tracking the calling patterns of the mobile device, and comparing these patterns to historical calling and mobility patterns of the user's mobile device and/or historical calling and mobility patterns of an average user within a same demographic group as the user of the mobile device. When there is a decline in the communications of the mobile device and a decline in the movement of the mobile device, it may be determined that the user is showing signs of depression, and when the declines are large enough, a determination may then be made that the user is likely suffering from depression.

The present disclosure also provides a transformation of data, e.g., mobile device usage data, including device location tracking data and call detail record data, is transformed into a calling pattern and a mobility pattern of the mobile device. The calling pattern and mobility pattern are also transformed into a depression score (another form of data), which can then be used to determine whether a user of the mobile device is likely suffering from depression. Finally, embodiments of the present disclosure improve the functioning of computing devices, e.g., a server or a mobile device. Namely, no server or mobile device is presently known to automatically detect and report a likelihood of depression based upon calling patterns and mobility patterns of the mobile device.

FIG. 3 depicts a high-level block diagram of a computing device suitable for use in performing the functions described herein. As depicted in FIG. 3, the system 300 comprises one or more hardware processor elements 302 (e.g., a central processing unit (CPU), a microprocessor, or a multi-core processor), a memory 304 (e.g., random access memory (RAM) and/or read only memory (ROM)), a module 305 for detecting a likelihood of depression, and various input/output devices 306 (e.g., storage devices, including but not limited to, a tape drive, a floppy drive, a hard disk drive or a compact disk drive, a receiver, a transmitter, a speaker, a display, a speech synthesizer, an output port, an input port and a user input device (such as a keyboard, a keypad, a mouse, a microphone and the like)). Although only one processor element is shown, it should be noted that the computing device may employ a plurality of processor elements. Furthermore, although only one computing device is shown in the figure, if the method 200 as discussed above is implemented in a distributed or parallel manner for a particular illustrative example, i.e., the steps of the method, or the entire method is implemented across multiple or parallel computing devices, then the computing device of this figure is intended to represent each of those multiple computing devices.

Furthermore, one or more hardware processors can be utilized in supporting a virtualized or shared computing environment. The virtualized computing environment may support one or more virtual machines representing computers, servers, or other computing devices. In such virtualized virtual machines, hardware components such as hardware processors and computer-readable storage devices may be virtualized or logically represented.

It should be noted that the present disclosure can be implemented in software and/or in a combination of software and hardware, e.g., using application specific integrated circuits (ASIC), a programmable gate array (PGA) including a Field PGA, or a state machine deployed on a hardware device, a computing device or any other hardware equivalents, e.g., computer readable instructions pertaining to the method discussed above can be used to configure a hardware processor to perform the steps, functions and/or operations of the above disclosed method. In one embodiment, instructions and data for the present module or process 305 for detecting a likelihood of depression (e.g., a software program comprising computer-executable instructions) can be loaded into memory 304 and executed by hardware processor element 302 to implement the steps, functions or operations as discussed above in connection with the illustrative method 200. Furthermore, when a hardware processor executes instructions to perform “operations”, this could include the hardware processor performing the operations directly and/or facilitating, directing, or cooperating with another hardware device or component (e.g., a co-processor and the like) to perform the operations.

The processor executing the computer readable or software instructions relating to the above described method can be perceived as a programmed processor or a specialized processor. As such, the present module 305 for detecting a likelihood of depression (including associated data structures) of the present disclosure can be stored on a tangible or physical (broadly non-transitory) computer-readable storage device or medium, e.g., volatile memory, non-volatile memory, ROM memory, RAM memory, magnetic or optical drive, device or diskette and the like. Furthermore, a “tangible” computer-readable storage device or medium comprises a physical device, a hardware device, or a device that is discernible by the touch. More specifically, the computer-readable storage device may comprise any physical devices that provide the ability to store information such as data and/or instructions to be accessed by a processor or a computing device such as a computer or an application server.

While various embodiments have been described above, it should be understood that they have been presented by way of example only, and not a limitation. Thus, the breadth and scope of a preferred embodiment should not be limited by any of the above-described exemplary embodiments, but should be defined only in accordance with the following claims and their equivalents.

What is claimed is:

1. A method for detecting a likelihood of depression of a user, comprising:
determining, by a processor, a calling pattern of a mobile device of the user during a first time period;
determining, by the processor, a mobility pattern of the mobile device of the user during the first time period;
detecting, by the processor, the likelihood of depression when the calling pattern of the mobile device during the first time period is indicative of a decline in communications as compared to a reference calling pattern and when the mobility pattern of the mobile device during the first time period is indicative of a decline in movement as compared to a reference mobility pattern; and
generating, by the processor, a warning message when the likelihood of depression is detected.

2. The method of claim 1, wherein the reference calling pattern is based upon historic calling records of a plurality of mobile devices during a second time period, and wherein the reference mobility pattern is based upon historic mobility records of the plurality of mobile devices during the second time period.

3. The method of claim 1, wherein the reference calling pattern is based upon historic calling records of the mobile device during a second time period, and wherein the reference mobility pattern is based upon historic mobility records of the mobile device during the second time period.

4. The method of claim 1, wherein the detecting comprises:
calculating a depression score, wherein the depression score is based on:
a comparison of the calling pattern of the mobile device during the first time period with the reference calling pattern; and
a comparison of the mobility pattern of the mobile device during the first time period with the reference mobility pattern; and
detecting the likelihood of depression when the depression score exceeds a threshold.

5. The method of claim 4, wherein the comparison of the calling pattern of the mobile device during the first time period with the reference calling pattern comprises:
comparing an average number of minutes of calls during a time interval of a second time period associated with the reference calling pattern with an average number of minutes of calls of the mobile device during a same time interval of the first time period.

6. The method of claim 5, wherein the average number of minutes of calls during the time interval of the second time period associated with the reference calling pattern comprises an average number of minutes of calls involving non-family members and non-work contacts during the time interval of the second time period associated with the reference calling pattern, and wherein the average number of minutes of calls of the mobile device during the same time interval of the first time period comprises an average number of minutes of calls involving non-family members and non-work contacts during the same time interval of the first time period.

7. The method of claim 4, wherein the comparison of the calling pattern of the mobile device during the first time period with the reference calling pattern comprises:
comparing an average number of calls during a time interval of a second time period associated with the reference calling pattern with an average number of calls of the mobile device during a same time interval of the first time period.

8. The method of claim 7, wherein the average number of calls during the time interval of the second time period associated with the reference calling pattern comprises an average number of calls involving non-family members and non-work contacts during the time interval of the second time period associated with the reference calling pattern, and wherein the average number of calls of the mobile device during the same time interval of the first time period comprises an average number of calls involving non-family members and non-work contacts during the same time interval of the first time period.

9. The method of claim 4, wherein the comparison of the mobility pattern of the mobile device during the first time period with the reference mobility pattern comprises:
comparing an average distance travelled during a time interval of a second time period associated with the reference mobility pattern with a distance travelled by the mobile device during a same time interval of the first time period.

10. The method of claim 4, wherein the comparison of the mobility pattern of the mobile device during the first time period with the reference mobility pattern comprises:
comparing a measure of time spent away from areas surrounding a home location and a work location during a time interval of a second time period associated with the reference mobility pattern with a measure of time spent away from areas surrounding the home location and the work location by the mobile device during a same time interval of the first time period.

11. The method of claim 1, wherein the first time period is associated with weekend days, wherein the reference calling pattern and the reference mobility pattern are associated with a second time period, and wherein the second time period is associated with the weekend days.

12. The method of claim 4, wherein the decline in the calling pattern and the decline in the mobility pattern are weighted parameters that contribute to the depression score.

13. The method of claim 12, wherein the depression score is further based on:
a comparison of a number of email messages associated with the mobile device during the first time period with an average number of email messages during a time interval of a second time period associated with the reference calling pattern.

14. The method of claim 12, wherein the depression score is further based on:
a comparison of a number of text messages associated with the mobile device during the first time period with a number of text messages during a second time period.

15. The method of claim 1, further comprising:
sending the warning message to a device associated with a medical professional; or
sending the warning message to a device associated with a caregiver of the user of the mobile device.

16. The method of claim 1, further comprising:
performing a remedial action in response to the detecting the likelihood of depression, wherein the remedial action comprises:
presenting a media item on the mobile device; or
sending a message to a contact of the user of the mobile device suggesting to the contact to communicate with the user.

17. A tangible computer-readable medium storing instructions which, when executed by a processor, cause the processor to perform operations for detecting a likelihood of depression of a user, the operations comprising:
determining a calling pattern of a mobile device of the user during a first time period;
determining a mobility pattern of the mobile device of the user during the first time period;
detecting the likelihood of depression when the calling pattern of the mobile device during the first time period is indicative of a decline in communications as compared to a reference calling pattern and when the mobility pattern of the mobile device during the first time period is indicative of a decline in movement as compared to a reference mobility pattern; and
generating a warning message when the likelihood of depression is detected.

18. The tangible computer-readable medium of claim 17, wherein the processor is deployed in the mobile device.

19. A device for detecting a likelihood of depression of a user, comprising:
a processor; and
a computer-readable medium storing instructions which, when executed by the processor, cause the processor to perform operations, the operations comprising:
determining a calling pattern of a mobile device of the user during a first time period;
determining a mobility pattern of the mobile device of the user during the first time period;
detecting the likelihood of depression when the calling pattern of the mobile device during the first time period is indicative of a decline in communications as compared to a reference calling pattern and when the mobility pattern of the mobile device during the first time period is indicative of a decline in movement as compared to a reference mobility pattern; and
generating a warning message when the likelihood of depression is detected.

20. The device of claim 19, wherein the processor is deployed in an application server of a communication network.
